(19) United States
(12) Patent Application Publication
(10) Pub. No.: US 2008/0058708 A1
(43) Pub. Date: Mar. 6, 2008

(54) Phacoemulsification Needle

(76) Inventor: Takayuki Akahoshi, Tokyo (JP)

Correspondence Address: JERRY A. SCHULMAN, 1 S. 376 SUMMIT AVENUE, COURT C, OAKBROOK TERRACE, IL, 60181, US

(21) Appl. No.: 11/461,747

(22) Filed: Aug. 1, 2006

Related U.S. Application Data
(60) Provisional application No. 60/704,556, filed on Aug. 2, 2005.
(60) Provisional application No. 60/725,129, filed on Oct. 8, 2005.
(60) Provisional application No. 60/754,450, filed on Dec. 28, 2005.

(51) Int. Cl. A61N 7/00

(52) U.S. Cl. 604/22

(57) ABSTRACT

A phacoemulsification needle uses a variety of tip shapes to increase efficiency during surgery. In several variations the tips are angled with respect to the needle body. Inb others, various cross-sectional shapes are used. Angling the tip opening is another configuration used.

### PRIORITY

This application claims priority from application Ser. No. 60/704,556, filed Aug. 2, 2005, application Ser. No. 60/725,129, filed Oct. 8, 2005 and application Ser. No. 60/754,450, filed Dec. 28, 2005, all of which are incorporated herein by reference.

### FIELD OF THE INVENTION

This disclosure relates to surgical instruments and surgical techniques used in eye surgery and more particularly, to phacoemulsification apparatus and methods for their use.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art.

Needles and tips of varying configurations are well known. A particular shape for a tip or needle is often dictated by the type of handpiece with which the needle is to be used.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

Known phacoemulsification needles and tips are designed for use with handpieces that vibrate the needle longitudinally at relatively low frequencies. In addition to longitudinal vibration, the NeoSoniX handpiece has a rotational or torsional oscillation vibration frequency of about 100 cycles/second. There is a new generation of handpiece that provides torsional oscillation of the phacoemulsification tip at frequencies of about 32,000 cycles/second. At such higher oscillation rates there is reduced sticking of nuclear fragments at the aspiration port. However, to be effective at such higher oscillation rates a phacoemulsification needle tip must be relatively light as compared to present tips while still providing a sufficiently large surface and aspiration port to efficiently emulsify and aspirate fragments of the nucleus.

I have determined that good results can be achieved using high-speed handpieces if the phacoemulsification tip is made to be lighter by changing the shape of the tip and removing portions of the tip material. I have also determined that better results are achieved if the tip is angled with respect to the needle axis. I have also determined that roughening the exterior surface of the tip enhances the emulsification effect.

In accordance with these criteria I have designed a series of tips that are reduced in weight without sacrificing size and have included specially roughened surfaces to enhance the emulsifying effect created by the torsional handpiece. Such tips and tip design criteria can be used for handpieces that combine the torsional and longitudinal vibratory motion.

While the following describes a preferred embodiment or embodiments of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

In accordance with a preferred embodiment of the apparatus a phacoemulsification needle is provided for use with a high-frequency torsional phacoemulsification handpiece with the needle having a needle tip angled with respect to the longitudinal axis of the needle and which has an oval cross-section and mouth communicating with the hollow passage formed through the needle.

In a second preferred embodiment, the exterior surfaces of the tip are roughened while the mouth of the tip is polished to a mirror finish.

In yet another embodiment of the present invention, the tip is formed in a “catfish” configuration wherein portions of the tip extending from the tip mouth are removed.

In yet another embodiment of the present invention, the tip is formed as a section of a sphere with opposed faces of the sphere flattened and roughened while the remaining portions of the tip are mirror polished.

In yet another embodiment the tip is formed with a triangular cross-section and at least one of the triangle sides is polished while the remaining side(s) are roughened.

In yet another embodiment the tip is flattened and has an elliptical cross-section and mouth.

In yet another embodiment the tip is formed with a generally squared-off or rectangular cross-section.

In yet another embodiment the tip is formed coaxially with the needle body and has a generally flattened shape.

### BRIEF DESCRIPTION OF THE DRAWINGS

These and further aspects of the present invention will be best understood by reference to the accompanying drawings wherein:

FIG. 1 is a drawing showing prior art oval- and square-shaped tips;

FIG. 2 is a drawing showing several prior art needle cross-sectional configurations;

FIG. 3 is a lateral view of a phacoemulsification needle embodying the present apparatus;

FIG. 4 is a lateral detail of the needle shown in FIG. 3;

FIG. 5 is an end view of the mouth of the needle tip shown in FIG. 4;

FIG. 6 is a partial sectional view along line 6-6 of FIG. 5;

FIG. 7 is a partial elevational view of a second embodiment of the needle and tip;

FIG. 8 is a view along 8-8 of FIG. 7;

FIG. 9 is a partial elevational view of a third embodiment of the needle and tip;

FIG. 10 is a view along 10-10 of FIG. 9;

FIG. 11 is a partial elevational view of a fourth embodiment of the needle and tip;

FIG. 12 is a view along line 12-12 of FIG. 11;

FIG. 13 is a partial elevational view of a fifth embodiment of the needle and tip;

FIG. 14 is a view along line 14-14 of FIG. 13;

FIG. 15 is a partial elevational view of a sixth embodiment of the needle and tip;

FIG. 16 is a partial elevational view of a seventh embodiment of the needle and tip;

FIG. 17 is a view along line 17-17 of FIGS. 15 and 16;

FIG. 18 is a partial elevational view of a prior art high-frequency tip;

FIG. 19 is a view along line 19-19 of FIG. 18;

FIG. 20 is a partial elevational view of an eighth embodiment of the needle and tip; and

FIG. 21 is a view along line 21-21 of FIG. 20.

### DETAILED DESCRIPTION OF THE INVENTION

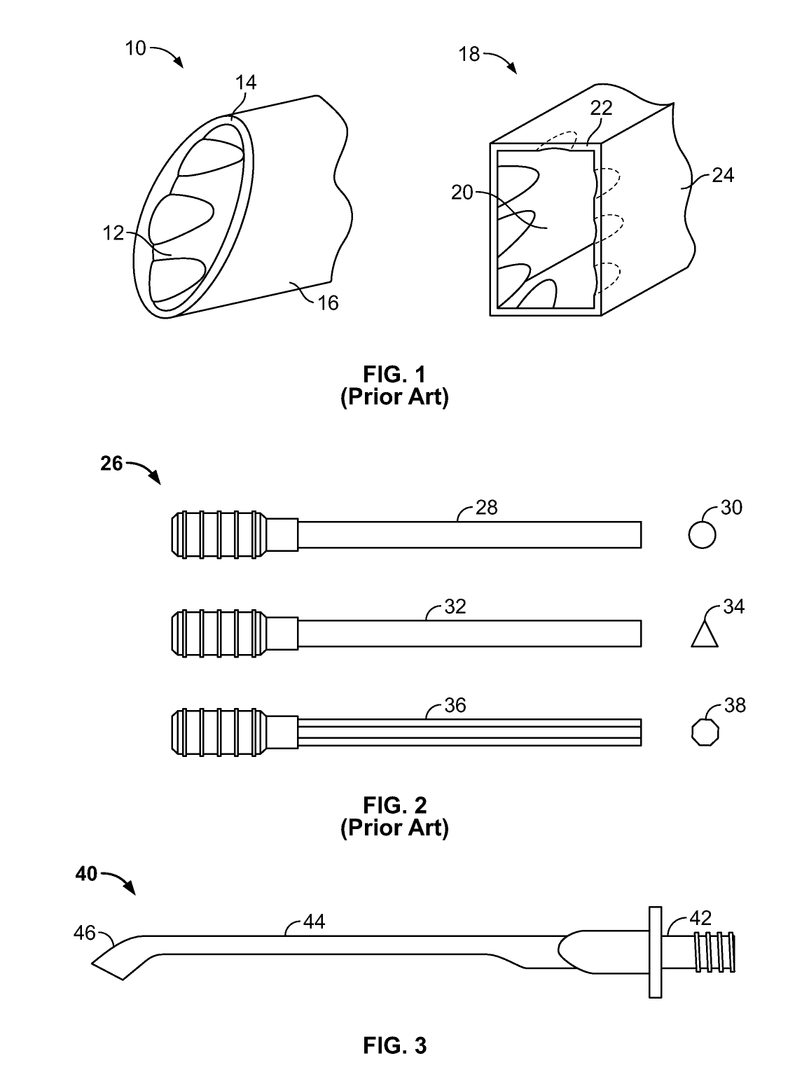

Referring now to FIG. 1, the numeral 10 indicates generally a prior art phacoemulsification needle tip as shown in U.S. Pat. No. 6,007,555. Needle 10 terminates in a mouth 12 defined by a lip 14 at the end of needle body 16, with lip 14 and needle body 16 formed as having an oval cross-section configuration.

Referring to FIG. 1, the numeral 18 indicates generally a prior art phacoemulsification needle tip from U.S. Pat. No. 6,007,555, having a mouth 20 defined by a lip 22 at the end of needle 24. The cross-sectional configuration of needle 18 and mouth 20 is a rectangle.

Referring now to FIG. 2, the numeral 26 identifies several prior art phacoemulsification needles as described in U.S. Pat. No. 5,725,495, with needle 28 having a circular cross-section as shown at 30, needle 32 having a triangular cross-section as shown at 34 and needle 36 having an octagonal cross-section as shown at 38.

Referring now to FIG. 3, the numeral 40 indicates generally a phacoemulsification needle embodying certain aspects of the present invention. Needle 40 has a mount 42 enabling it to be attached to a phacoemulsification handpiece (not shown). Needle 40 has an elongate body 44 extending from mount 42 and terminating in a needle tip 46. As is common with phacoemulsification needles of this type, a hollow passageway is formed extending from mount 42 through tip 46 and is used to aspirate emulsified lens fragments during surgery

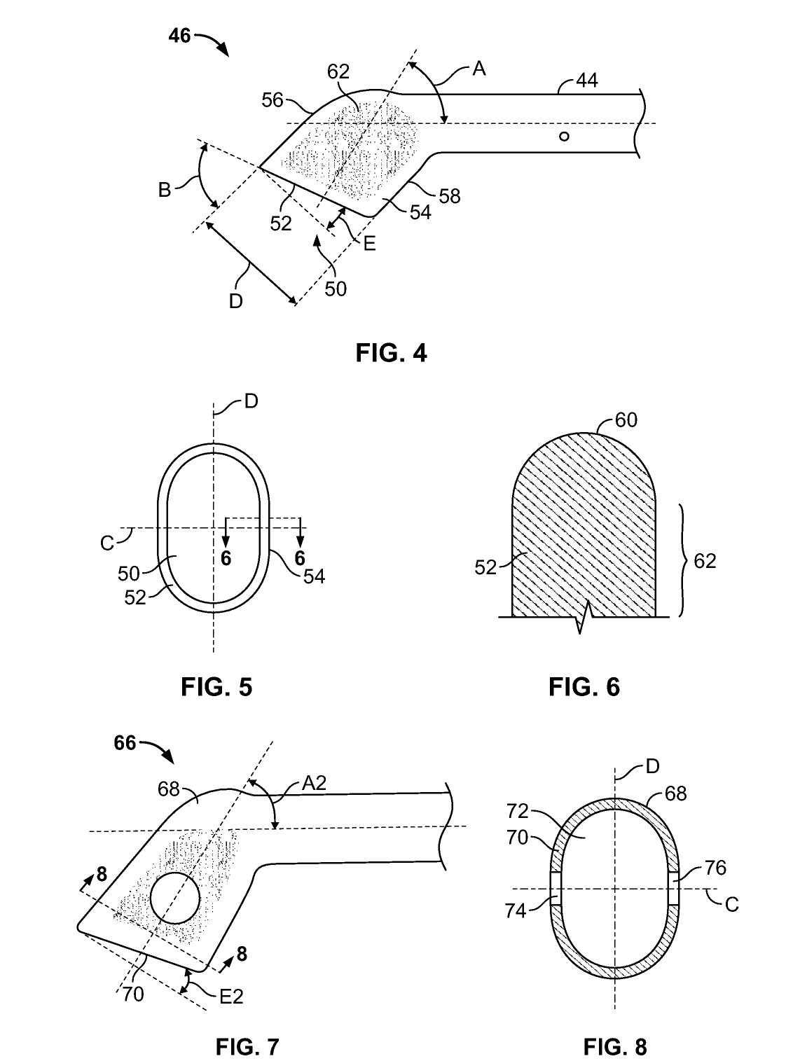

Referring now to FIG. 4, an enlarged view of tip 46 is shown. Tip 46 is offset from the axis 48 of body 44 by a tip-body offset angle A, preferably about 10-15°.

As seen in FIG. 4, tip 46 is flared and somewhat bell-shaped and has a mouth 50 defined by a lip 52. Mouth 50 is best seen in FIG. 5. Tip 46 has a tip body 54 comprising the outer surface of tip 46 extending from lip 52 to where tip 46 meets body 44.

As seen in FIG. 4, tip body 54 is preferably somewhat flattened and preferably foreshortened with lead edge 56 being longer than trailing edge 58 and with lead edge 56 meeting lip 52 at an angle B, preferably about 65°. As seen in FIG. 5, mouth 50 is generally oval in shape as is lip 52.

As seen in FIG. 6, a portion 60 of lip 52 is polished to a mirror finish to allow lip 52 and thereby tip 46 to pass through an incision without snagging the incised tissue.

The mirror polishing of lip 52 also provides added protection against the rupture of the posterior capsule of the eye should tip 52 come in contact therewith.

Referring now to FIGS. 4 and 6, a portion 62 of tip body 54 is roughened, as by sandblasting or the like, to enhance the emulsification effect of needle 46 during the phacoemulsification process.

In a preferred embodiment and as seen in FIGS. 4 ad 5, tip 46 is about 1.5 mm along its longest dimension while, as seen in FIG. 5, mouth 50 is about 1.8 mm at its major axis D and 1.0 mm at its minor axis C, with a wall thickness of about 0.1 mm. As seen in FIGS. 4 and 5, major axis D lies generally in or parallel to a plane that includes tip 46, body 44 and angle A. This will be referred to as the “tip-body” plane. As also seen in FIG. 4 mouth 50 is also angled laterally in the tip-body plane at a head-offset angle E of about 25°.

When used in phacoemulsification, mouth 50, as the terminus of the hollow channel formed through needle 40 allows lens and other tissue fragments created by the phacoemulsification process to be aspirated through needle 40.

Referring now to FIG. 7, the numeral 66 identifies generally a phacoemulsification needle tip having an outer surface 68 terminating in a lip 70. As seen in FIG. 8, lip 70 and outer surface 68 define an oval mouth 72 as described above in connection with the first preferred embodiment. As described above, tip 66 has a a tip-body offset angle A2, preferably about 10-15° and a head offset angle E2 of about 25°.

Referring now to FIG. 8, a pair of lateral ports 74, 76 are formed opposite one another and extending through tip 66. As described hereinabove, lip 70 is mirror polished while the outer surface 68 is roughened by sandblasting.

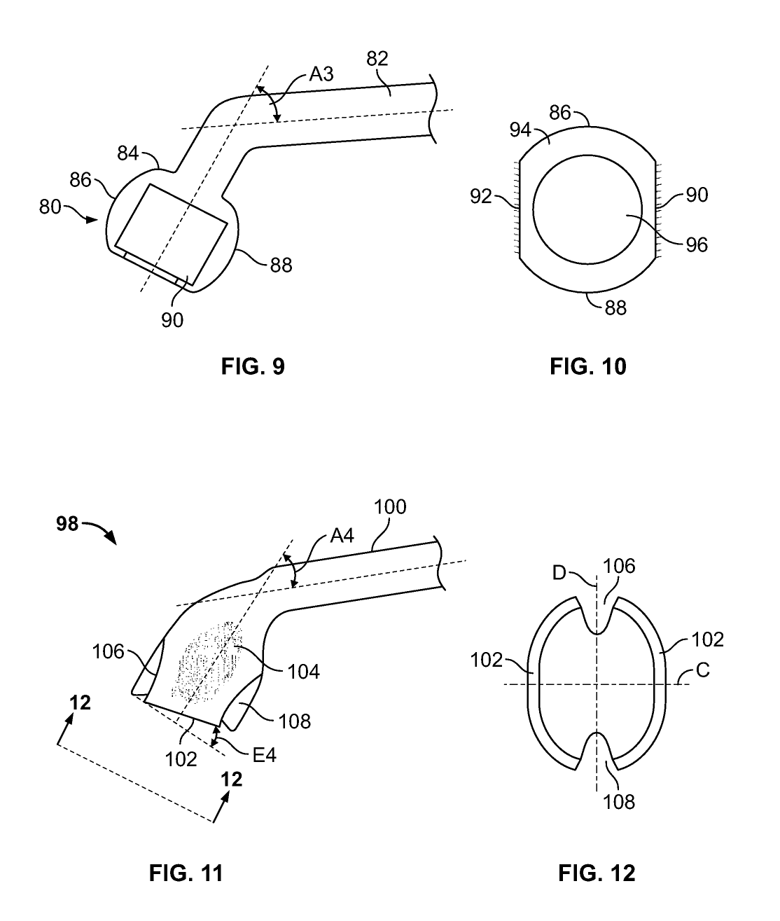

Referring now to FIG. 9, a third embodiment of the present invention is shown wherein the numeral 80 identifies a needle tip. As described above, tip 80 has a a tip-body offset angle A3, preferably about 10-15°. Tip 80 terminates in a rounded body 84 having a pair of opposed, curved sides 86, 88 and a pair of opposed flattened sides 90, 92 as illustrated in FIG. 10. Sides 86, 88, 90 and 92 terminate in and form a lip 94 extending about a circular mouth 96. As described hereinabove, surfaces 90, 92 are roughened while curved surfaces 86, 88 are mirror polished.

Referring now to FIG. 11, the numeral 98 identifies a phacoemulsification tip extending from needle body 100. As described above, tip 98 has a a tip-body offset angle A4, preferably about 10-15° and a head offset angle E4 of about 25°. Tip 98 terminates at a lip 102 which, as described above, is mirror-polished. Tip outer surface 104 is roughened, as by sandblasting or the like and has a pair of opposed cut-outs 106, 108 formed thereon. As seen in FIG. 12, each cutout 106, 108 begins at lip 102 and extends along tip 98 toward body 100. Tip 98 is thereby lightened by the material removed to form cutouts 106, 108 while retaining the rigidity needed for tip 98 to function.

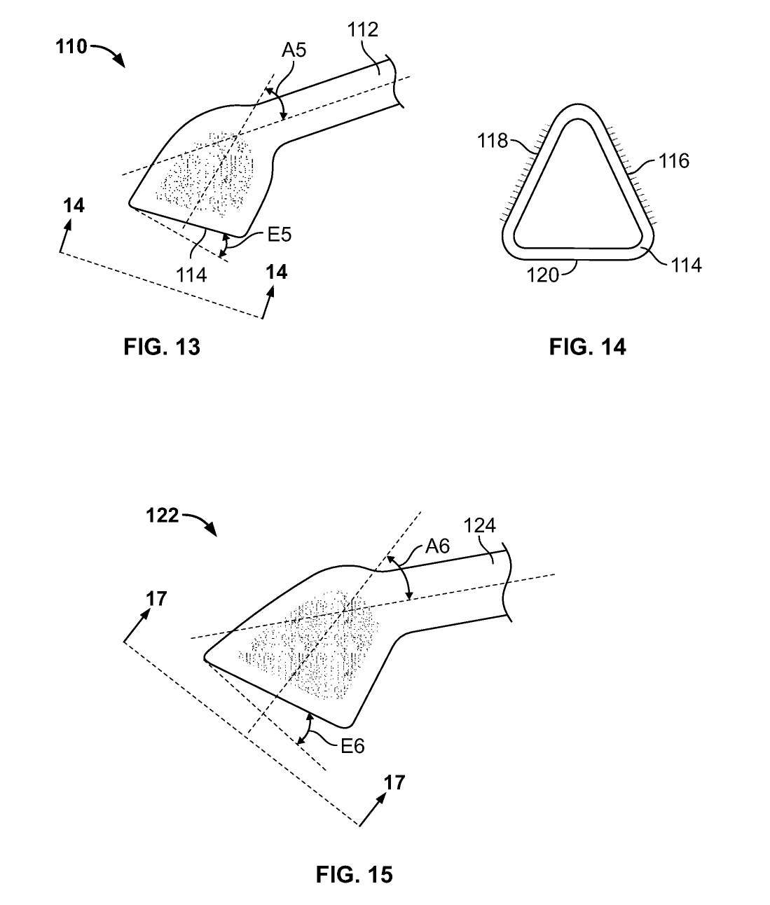

Referring now to FIG. 13, the numeral 110 identifies a phacoemulsification tip extending from needle body 112 . As described above, tip 110 has a a tip-body offset angle A5, preferably about 10-15° and a head offset angle E5 of about 25°. As seen in FIG. 14, tip 110 is triangular in cross-sectional configuration, terminating in a lip 114 and having sidewalls 116, 118 and 120, each of which preferably taper upward towards body 112. In the embodiment shown, the outer surface of sidewall 120 is mirror-polished, while the outer surfaces of sidewalls 116 and 118 are roughened as described above.

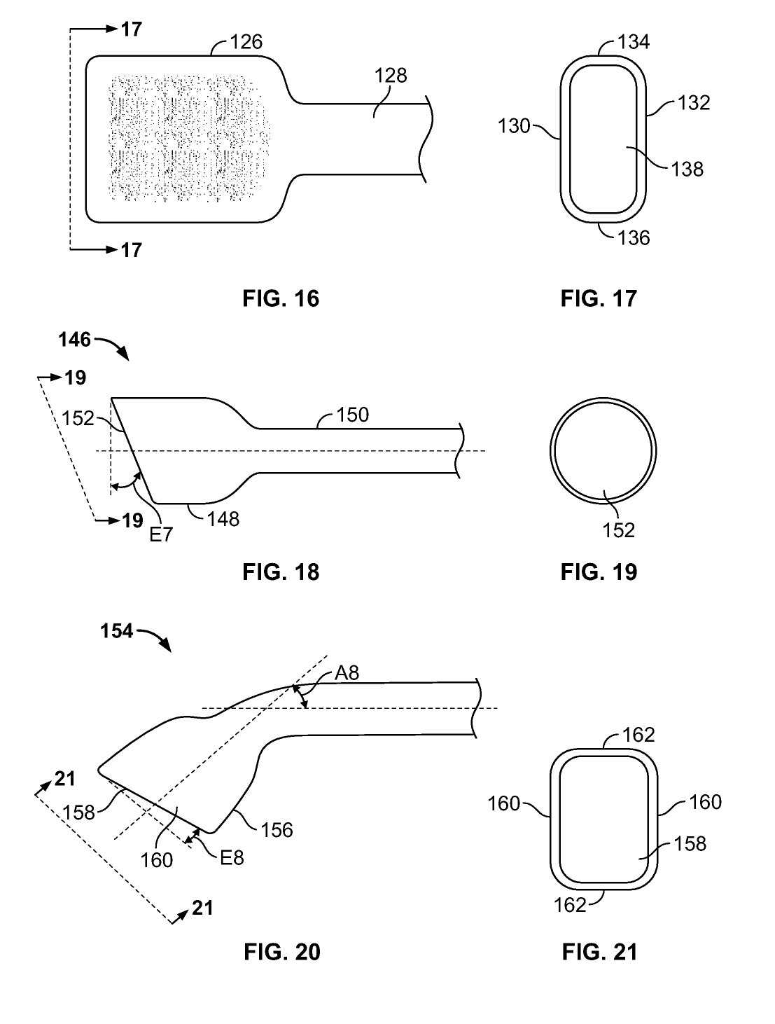

In FIGS. 15, 16 and 17 phacoemulsification tips embodying the present invention are shown having flattened profiles. In FIG. 15, tip 122, as described above, has a a tip-body offset angle A6, preferably about 10-15° and a head offset angle E6 of about 25°.

In FIG. 17, tip 126 is coaxial with body 128. Tips 122, 126 are formed with a flattened oval-type configuration as seen in FIG. 17, with lip segments 130, 132 generally parallel to each other, and with lip segments 134, 136 generally parallel to each other to form a “racetrack-like” mouth 138. As described above, the outer surfaces of tips 122, 126 are roughened, and lip segments 130, 132, 134 and 136 are mirror-polished.

Referring now to FIG. 18 a known high-frequency needle 146 is shown having a tip 148, a body 150 and a mouth 152. As described above, tip 146 has a head offset angle E7 of about 25°. Tip 148 and body 150 are “coaxial”, meaning that there is little or no tip-body offset angle. In addition, as seen in FIG. 19, mouth 152 is a round opening.

Referring to FIG. 20, an additional embodiment of the present apparatus is shown wherein needle 154 has a a tip-body offset angle A8, preferably about 10-15 and a head offset angle E8 of about 25° Needle 154 has a tip 156 terminating in a mouth 158 which, as seen in FIG. 21 has a generally rectangular shape with opposed walls 160 being longer in dimension than opposed walls 162. As seen in FIG. 20, walls 160 are in the “tip-body” plane, meaning that the longer dimensions of mouth 158 are generally parallel to the bend formed by the head offset angle.

Angling the head appears to maximize the phacoemulsificiation effect created by the high-frequency handpieces described above and it appears that an angle of 10-15° produces very satisfactory results, with larger angles used for relatively smaller or lighter heads, while the smaller angles are used with relatively larger or heavier heads. Larger heads with larger mouth openings create the most efficient results.

While the tip configurations depicted herein generally show each non-circular tip mouth positioned to place either a major or minor axis parallel to the tip-body plane it is also contemplated that either such axis can lie in a plane that is at an angle to the tip-body plane.

What is claimed is:

1. A phacoemulsification needle, said needle comprising:
a hollow, elongate needle body,
said body terminating in a needle tip;
said tip having an exterior surface and a mouth,
said mouth terminating in and defined by a lip;
said tip formed at an angle with respect to said body.

2. The apparatus as recited in claim 2 wherein said angle is from about 10° to about 15°.

3. The apparatus as recited in claim 2 wherein said tip has a generally flattened and generally oval cross-sectional shape.

4. The apparatus as recited in claim 3 wherein said mouth is generally oval.

5. The apparatus as recited in claim 4 wherein said lip is polished to be smooth.

6. The apparatus as recited in claim 3 wherein at least a portion of the exterior surface of said tip is roughened.

7. The apparatus as recited in claim 3 wherein said oval mouth has a major axis and a minor axis;
said major axis intersecting said tip at a leading edge and a trailing edge;
said mouth being angled at about 25° from said leading edge to said trailing edge.

8. The apparatus as recited in claim 2 wherein said tip has a generally oval cross-sectional shape.

9. The apparatus as recited in claim 8 wherein said mouth is generally oval.

10. The apparatus as recited in claim 9 wherein said lip is polished.

11. The apparatus as recited in claim 8 wherein at least a portion of the exterior surface of said tip is roughened.

12. The apparatus as recited in claim 9 wherein said oval mouth has a major axis and a minor axis;
said major axis intersecting said tip at a leading edge and a trailing edge;
said mouth being angled at about 25° from said leading edge to said trailing edge

13. The apparatus as recited in claim 8 wherein said tip has at least one port formed through said exterior surface.

14. The apparatus as recited in claim 2 wherein said tip has opposed first and second curved exterior surface portions and opposed first and second flattened exterior surface portions.

15. The apparatus as recited in claim 14 wherein said tip has a generally circular cylindrical passage formed therethrough.

16. The apparatus as recited in claim 14 wherein at least a portion of said first and second opposed curved surface portions are polished to be smooth.

17. The apparatus as recited in claim 14 wherein at least a portion of said first and second opposed flattened surface portions are roughened.

18. The apparatus as recited in claim 2 wherein said tip has a generally oval cross-sectional shape,
said tip having a major axis and a minor axis,
said major axis intersecting said tip at a leading edge and a trailing edge;
at least one cutout formed in said exterior surface communicating with said mouth.

19. The apparatus as recited in claim 18 wherein said tip has two opposed said cutouts

20. The apparatus as recited in claim 19 wherein said opposed cutouts are positioned at said leading and trailing edges.

21. The apparatus as recited in claim 2 wherein said tip has a triangular cross-sectional shape.

22. The apparatus as recited in claim 21 wherein said lip is polished to be smooth.

23. The apparatus as recited in claim 21 wherein at least a portion of the exterior surface of said tip is roughened.

24. The apparatus as recited in claim 21 wherein said mouth has a leading edge and a trailing edge;
said mouth being angled at about 25° from said leading edge to said trailing edge.

25. The apparatus as recited in claim 2 wherein said tip has a generally rectangular cross-sectional shape.

26. The apparatus as recited in claim 25 wherein said lip is polished to be smooth.

27. The apparatus as recited in claim 25 wherein at least a portion of the exterior surface of said tip is roughened.

28. The apparatus as recited in claim 25 wherein said mouth has a leading edge and a trailing edge;
said mouth being angled at about 25° from said leading edge to said trailing edge.

29. The apparatus as recited in claim 25 wherein said tip has a generally square cross-sectional shape.

30. A phacoemulsification needle, said needle comprising:
a hollow, elongate needle body,
said body terminating in a needle tip;
said tip having an exterior surface and a mouth,
said mouth terminating in and defined by a lip;
said mouth having a generally flattened rectangular cross-sectional shape.

31. A phacoemulsification needle, said needle comprising:
a hollow, elongate needle body,
said body terminating in a needle tip;
said tip having an exterior surface and a mouth,
said mouth terminating in and defined by a lip;
said mouth having a circular cross-sectional shape;
said mouth having a leading edge and a trailing edge;
said mouth being angled at about 25° from said leading edge to said trailing edge.
